(19) United States
(12) Patent Application Publication
(10) Pub. No.: US 2026/0118338 A1
(43) Pub. Date: Apr. 30, 2026

(54) METHOD FOR HIGH-THROUGHPUT DETERMINATION OF WHOLE WATER TOXICITY

(71) Applicant: Nanjing University, Nanjing (CN)

(72) Inventors: Haidong HU, Nanjing (CN); Kewei LIAO, Nanjing (CN); Hongqiang REN, Nanjing (CN)

(21) Appl. No.: 18/965,999

(22) Filed: Dec. 2, 2024

(30) Foreign Application Priority Data
Oct. 31, 2024 (CN) 202411537609.2

(51) Int. Cl. G01N 33/18; G01N 1/34; G01N 21/64; G01N 33/50

(52) U.S. Cl. CPC G01N 33/1866; G01N 1/34; G01N 21/6456; G01N 21/6486; G01N 33/5014

(57) ABSTRACT

A method for high-throughput determination of whole water toxicity, including: exposing test organisms in a wastewater sample for pollution, obtaining phenotypic feature data of the test organisms; constructing a toxicity matrix; and building a machine learning model, and in combination with the toxicity matrix, determining a whole toxicity of the wastewater sample.

### CROSS-REFERENCE TO RELATED APPLICATIONS

Pursuant to 35 U.S.C. § 119 and the Paris Convention Treaty, this application claims foreign priority to Chinese Patent Application No. 202411537609.2 filed Oct. 31, 2024, the contents of which, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, MA 02142.

### BACKGROUND

The disclosure relates to the field of water quality risk control, and more particularly to a method for high-throughput determination of whole water toxicity (WWT).

Wastewater contains a wide variety of pollutants, which have a direct toxic effect on organisms, and may combine with other pollutants to produce a composite toxic effect. The use of a single/specific pollutant concentration level or a single/specific pollutant toxicity effect value is difficult to accurately reflect the whole water toxicity, and may produce a large deviation. Commonly used methods at home and abroad to evaluate the whole water toxicity include the whole toxicity assessment method, toxicity identification assessment method, direct toxicity assessment method and so on. The whole toxicity assessment method involves exposing standard model organisms to gradient-diluted samples, and determining the acute toxicity values of the samples to the subject organisms under a fixed exposure time. However, these methods require a lot of time to determine the acute toxicity values through multiple exposure experiments in gradient-diluted samples, so it is difficult to realize rapid detection of toxic effects in a large number of wastewater samples in a short period of time. In addition, there is variability in the sensitivity and tolerance of the subject organisms used for toxicity testing to different pollutants and water qualities.

### SUMMARY

One objective of the disclosure is to provide a method for high-throughput determination of whole water toxicity, to solve the problems of conventional determination methods such as low detection throughput and the variability of the sensitivity and tolerance of subject organisms used for toxicity testing to different pollutants/water qualities.

The disclosure provides a method for high-throughput determination of whole water toxicity, the method comprising: exposing test organisms in a wastewater sample for pollution, obtaining phenotypic feature data of the test organisms; constructing a toxicity matrix; and building a machine learning model, and in combination with the toxicity matrix, determining a whole toxicity of the wastewater sample.

In a class of this embodiment, an exposure time of the test organisms in the wastewater sample for pollution is 24 hours.

In a class of this embodiment, prior to exposing the test organisms in the wastewater sample, the wastewater sample is pretreated.

In a class of this embodiment, the pre-treatment process of the wastewater sample comprises filtering the wastewater sample through a 0.22 μm membrane filter for aqueous solutions.

In a class of this embodiment, the test organisms are algae cells and fish gill cells.

In a class of this embodiment, the test organisms are selenastrum capricornutum cells and rainbow trout gill cells.

In a class of this embodiment, following exposure in the wastewater sample for pollution, the test organisms are extracted through multiple fluorescent staining, high-content automated imaging, and cellular morphological characterization, to obtain the phenotypic feature data of the test organisms.

In a class of this embodiment, multiple fluorescent stains for algae cell staining comprise Hoechst 33342 dye, concanavalin A/Alexa Fluor 488 dye, SYTO 14 dye, phalloidin/Alexa Fluor 568 and wheat-germ agglutinin/Alexa Fluor 555 dye; multiple fluorescent stains for gill cell staining comprise Hoechst 33342 dye, concanavalin A/Alexa Fluor 488 dye, SYTO 14 dye, phalloidin/Alexa Fluor 568 and wheat-germ agglutinin/Alexa Fluor 555 dye, and MitoTracker Deep Red dye.

In a class of this embodiment, the high-content automated imaging adopts a high content cell imaging and analysis system for high throughput automatic acquisition of subcellular structure images of algae cells and fish gill cells of 4-8 parallel experiments inoculated in a well plate.

In a class of this embodiment, the image acquisition conditions of the high content cell imaging and analysis system are as follows: each well in the orifice plate is equipped with 9 (3×3) imaging field points, which are merged using 2×2 pixels. Each point automatically captures 5-color fluorescence channel images and 3 bright field channel images from different z-axis focal points. The subcellular structure images of algae cells are obtained using a 63× immersion objective lens, and the subcellular structure images of fish gill cells are obtained using a 20× immersion objective lens; the excitation/emission wavelengths of the 5-color fluorescence channel used for automatic imaging of algae cells are DNA 376-398 nm/417-477 nm, ER 442-502 nm/503-538 nm, RNA 491-571 nm/573-613 nm, AGP 502-622 nm/622-662 nm, Cy5 588-668 nm/652-732 nm; the excitation/emission wavelengths of the 5-color fluorescence channel used for automatic imaging of fish gill cells are DNA 376-398 nm/417-477 nm, ER 442-502 nm/503-538 nm, RNA 491-571 nm/573-613 nm, AGP 502-622 nm/622-662 nm, Mito 588-668 nm/672-712 nm.

In a class of this embodiment, acquisition of the phenotypic feature data of the test organisms comprises positioning cells, nuclei, and cytoplasm in each image, and a quality of each image satisfies the following conditions: image intensity mean value of 10-240, image focus score>0.5, image edge front standard deviation<0.2, cell area of 50-500, cell debris hole number<5, cell density>50, and cell nucleus staining clarity>1.5; a gray level co-occurrence matrix for texture feature analysis is used to calculate a morphology, intensity, texture, brightness, average grayscale, a minimum distance between cells, adjacency values, and clustering degree, to obtain morphological feature items of each cell and an arithmetic mean of morphological feature values corresponding to the morphological feature items of each cell.

In a class of this embodiment, 5797 morphological features of each cell are obtained.

In a class of this embodiment, the toxicity matrix comprises phenotypic feature data of algae cells and fish gill cells acquired through clustering arrangement after operations of filtering feature items and standardizing feature values; operation of filtering feature items is to exclude collinear crossing feature items, and retain feature items whose eigenvalues are not equal to 0; the operation of standardizing feature values adopts a Z-Score method and a maximum-minimum method; the clustering arrangement is to classify and integrate the feature items according to corresponding subcellular structure of the feature items, and the classification comprises algae cell DNA, algae cell endoplasmic reticulum, algae cell nucleosomes and cytoplasmic RNA, algae cell actin with Golgi apparatus and plasma membrane, algae cell chloroplasts, algae cell bright field, fish gill DNA, fish gill endoplasmic reticulum, fish gill nucleosomes and cytoplasmic RNA, fish gill actin with Golgi apparatus and plasma membrane, fish gill cell mitochondria, and fish gill cell bright field.

In a class of this embodiment, the machine learning model is built based on acute toxicity effect values and the phenotypic feature data of algae cells and acute toxicity effect values and the phenotypic feature data of the fish gill cells, through a random forest model, XGBoost algorithmic model, Lasso regression algorithmic model, content-based recommendation algorithmic model, or support vector machine model.

In a class of this embodiment, determining a whole toxicity of the wastewater sample comprises performing feature dimensionality reduction on the constructed toxicity matrix using partial least squares discriminant analysis, to obtain feature variables of whole water toxicity, and substituting the feature variables of whole water toxicity into the machine learning model to obtain the whole toxicity of the wastewater sample; the whole toxicity of the wastewater sample is acute toxicity effect values caused by the wastewater sample, and expressed as a 10% effect concentration (EC₁₀).

In a class of this embodiment, the feature variables of the whole water toxicity have 12 items.

The following advantages are associated with the method for high-throughput determination of whole water toxicity of the disclosure.
1. The method for high-throughput determination of whole water toxicity of the disclosure solves the problems of complex detection steps, long time consumption, and low detection flux in existing methods.
2. Based on high-content cellular imaging technology and machine learning model, the method of the disclosure automatically captures the toxic effects of the whole water toxicity of the wastewater samples at the cellular and subcellular structure level, and at the same time accurately quantifies the acute toxicity effects of the wastewater samples, thus greatly simplifying the determination steps, and guaranteeing the comprehensiveness and accuracy of the results.
3. The disclosure utilizes the different response characteristics of algae cells and fish gill cells to the toxic effects of wastewater samples to construct a toxicity matrix, which reduces the selectivity of the existing methods for specific wastewater samples, enhances the universality of the high-throughput determination of the whole water toxicity for various types of wastewater samples, and improves the practical applicability of the method.

### BRIEF DESCRIPTION OF THE DRAWINGS

FIG. 1 is a flow chart of a method for high-throughput determination of whole water toxicity in accordance with one embodiment of the disclosure;

FIG. 2 shows subcellular structural images of algae cells and gill cells in Example 1 of the disclosure;

FIG. 3 shows a toxicity matrix constructed from phenotypic feature data of algal and gill cells in Example 1 of the disclosure;

FIG. 4 shows the whole water toxicity of the wastewater samples from Plant B in Example 2 of the disclosure; and

FIG. 5 shows the whole water toxicity of effluents from plants C, D and E in Example 3 of the disclosure.

### DETAILED DESCRIPTION

To further illustrate the disclosure, embodiments detailing a method for high-throughput determination of whole water toxicity are described below. It should be noted that the following embodiments are intended to describe and not to limit the disclosure.

### Example 1

The application object of the example was an influent sample of a municipal sewage treatment plant in Jiangsu Province. The daily processing capacity of Plant A was 80000 cubic meters per day, with an influent COD of 254.0 mg/L, total nitrogen of 29.27 mg/L, and total phosphorus of 2.07 mg/L. A method for high-throughput determination of whole toxicity of the influent sample is as follows:
1. The influent sample from Plant A was filtered through a 0.22 μm membrane filter for aqueous solutions.
2. Algae cells of Selenastrum capricornutum and gill cells of rainbow trout were used as test organisms, and were exposed for 24 h in the filtered influent sample. Hoechst 33342 dye, concanavalin A/Alexa Fluor 488 dye, SYTO 14 dye, phalloidin/Alexa Fluor 568 dye, and wheat gene agglutinin/Alexa Fluor 555 dye were mixed to prepare a first multiple fluorescent staining agent, and the algae cells were exposed in the first multiple fluorescent staining agent. Hoechst 33342 dye, concanavalin A/Alexa Fluor 488 dye, SYTO 14 dye, phalloidin/Alexa Fluor 568, wheat-germ agglutinin/Alexa Fluor 555 dye, and MitoTracker Deep Red dye were mixed to prepare a second multiple fluorescent staining agent, and the gill cells were exposed in the second multiple fluorescent staining agent. A high content cell imaging and analysis system was adopted for high throughput automatic acquisition of subcellular structure images of algae cells and fish gill cells of 6 parallel experiment groups inoculated in a well plate. The subcellular structure images of algae cells were obtained using a 63× immersion objective lens, and the subcellular structure images of fish gill cells were obtained using a 20× immersion objective lens; the excitation/emission wavelengths of the 5-color fluorescence channel used for automatic imaging of algae cells were DNA 376-398 nm/417-477 nm, ER 442-502 nm/503-538 nm, RNA 491-571 nm/573-613 nm, AGP 502-622 nm/622-662 nm, Cy5 588-668 nm/652-732 nm; the excitation/emission wavelengths of the 5-color fluorescence channel used for automatic imaging of fish gill cells were DNA 376-398 nm/417-477 nm, ER 442-502 nm/503-538 nm, RNA 491-571 nm/573-613 nm, AGP 502-622 nm/622-662 nm, Mito 588-668 nm/672-712 nm. Each well in the orifice plate was equipped with 9 (3×3) imaging field points, which were merged using 2× 2 pixels. Each point automatically captures 5-color fluorescence channel images and 3 bright field channel images from different z-axis focal points. The acquisition of the phenotypic feature data of the test organisms with automatically captured images comprises positioning cells, nuclei, and cytoplasm in each image, and a quality of each image satisfies the following conditions: image intensity mean value of 10-240, image focus score>0.5, image edge front standard deviation<0.2, cell area of 50-500, cell debris hole number<5, cell density>50, and cell nucleus staining clarity>1.5; a gray level co-occurrence matrix for texture feature analysis was used to calculate a morphology, intensity, texture, brightness, average grayscale, a minimum distance between cells, adjacency values, and clustering degree, to obtain 5797 morphological feature items of each cell and an arithmetic mean of morphological feature values corresponding to the morphological feature items of each cell.
3. The collinear crossing feature terms of the phenotypic feature data of the algae cells and gill cells were excluded, and the feature items whose eigenvalues were not equal to 0 were retained; the standardizing feature values comprised a Z-Score method and a maximum-minimum method; the clustering arrangement was to classify and integrate the feature items according to corresponding subcellular structure of the feature items, and corresponding categories comprise algae cell DNA, algae cell endoplasmic reticulum, algae cell nucleosomes and cytoplasmic RNA, algae cell actin with Golgi apparatus and plasma membrane, algae cell chloroplasts, algae cell bright field, fish gill DNA, fish gill endoplasmic reticulum, fish gill nucleosomes and cytoplasmic RNA, fish gill actin with Golgi apparatus and plasma membrane, fish gill cell mitochondria, and fish gill cell bright field, to construct the toxicity matrix.
4. The machine learning model was built based on acute toxicity effect values and the phenotypic feature data of algae cells and acute toxicity effect values and the phenotypic feature data of the fish gill cells through a random forest model; feature dimensionality reduction was carried out on the constructed toxicity matrix using partial least squares discriminant analysis, to obtain 12 feature variables of whole water toxicity, which were substituted into the machine learning model to obtain the whole toxicity of the influent sample of the plant A, expressed as a 10% effect concentration (EC₁₀).

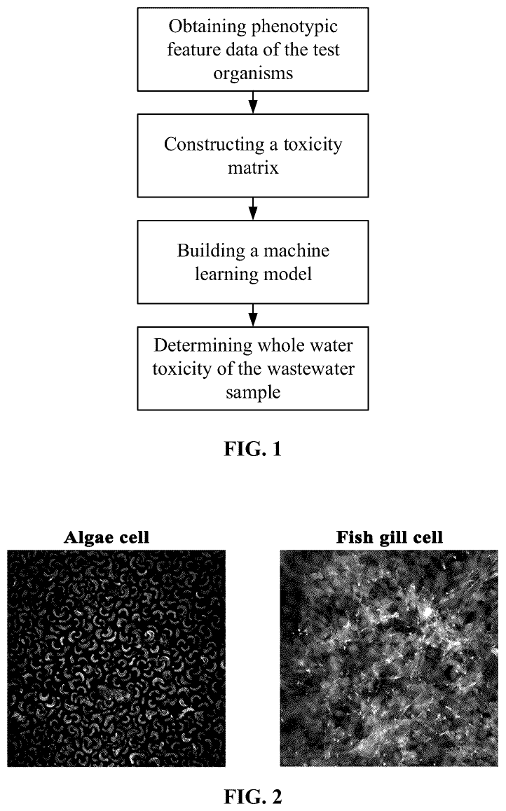

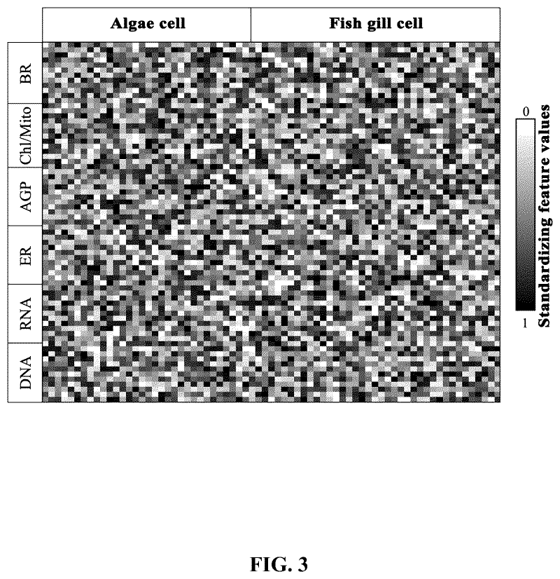

FIG. 2 shows the subcellular structural images of algae cells and gill cells in the influent sample of Plant A according to the method of the example. As shown in FIG. 3, the cellular morphological features were extracted to obtain the cellular phenotypic feature data of algae cells and gill cells for construction of the toxicity matrix. As shown in Table 1, the least partial squares discriminant analysis method was used to reduce the dimensionality of the toxicity matrix, and 12 whole toxicity feature variables associated with water quality were obtained, which were substituted into the machine learning model to obtain the whole water toxicity of the influent from Plant A, i.e., 55.2%.

TABLE 1
12 feature variables of whole water toxicity obtained in Example 1

| Feature variables of whole water toxicity | Value |
|---|---|
| DNA_1 | −20.173203 |
| DNA_2 | 3.73463273 |
| RNA_1 | −15.385017 |
| RNA_2 | 4.7597349 |
| ER_1 | −21.962306 |
| ER_2 | 2.68094709 |
| AGP_1 | −18.088792 |
| AGP_2 | 2.38816954 |
| Chl | −18.7958 |
| Mito | 4.47955748 |
| BR_1 | −17.150802 |
| BR_2 | 7.18030231 |

### Example 2

Unlike Example 1, the application object of the example was a municipal wastewater treatment plant B in Southwest China, which includes wastewater samples from the influent, aeration and sand sedimentation tank, anoxic tank, aerobic tank, secondary sedimentation tank, sand filter, disinfection tank, and effluent, and the daily capacity of the plant B was 450,000 m³/day, and the influent was 241.1 mg/L of COD, 27.02 mg/L of total nitrogen, and 2.94 mg/L of total phosphorus; the effluent was 55.40 mg/L of COD, 10.37 mg/L of total nitrogen, and 0.38 mg/L of total phosphorus. A method for high-throughput determination of whole toxicity of the influent sample is as follows:
1. Eight influent samples from Plant B were filtered through a 0.22 μm membrane filter for aqueous solutions, respectively.
2. Algae cells of Selenastrum capricornutum and gill cells of rainbow trout were used as test organisms, and were exposed for 24 h in the filtered influent sample. Hoechst 33342 dye, concanavalin A/Alexa Fluor 488 dye, SYTO 14 dye, phalloidin/Alexa Fluor 568 dye, and wheat gene agglutinin/Alexa Fluor 555 dye were mixed to prepare a first multiple fluorescent staining agent, and the algae cells were exposed in the first multiple fluorescent staining agent. Hoechst 33342 dye, concanavalin A/Alexa Fluor 488 dye, SYTO 14 dye, phalloidin/Alexa Fluor 568, wheat-germ agglutinin/Alexa Fluor 555 dye, and MitoTracker Deep Red dye were mixed to prepare a second multiple fluorescent staining agent, and the gill cells were exposed in the second multiple fluorescent staining agent. A high content cell imaging and analysis system was adopted for high throughput automatic acquisition of subcellular structure images of algae cells and fish gill cells of 4 parallel experiment groups inoculated in a well plate. The subcellular structure images of algae cells were obtained using a 63× immersion objective lens, and the subcellular structure images of fish gill cells were obtained using a 20× immersion objective lens; the excitation/emission wavelengths of the 5-color fluorescence channel used for automatic imaging of algae cells were DNA 376-398 nm/417-477 nm, ER 442-502 nm/503-538 nm, RNA 491-571 nm/573-613 nm, AGP 502-622 nm/622-662 nm, Cy5 588-668 nm/652-732 nm; the excitation/emission wavelengths of the 5-color fluorescence channel used for automatic imaging of fish gill cells were DNA 376-398 nm/417-477 nm, ER 442-502 nm/503-538 nm, RNA 491-571 nm/573-613 nm, AGP 502-622 nm/622-662 nm, Mito 588-668 nm/672-712 nm. Each well in the orifice plate was equipped with 9 (3×3) imaging field points, which were merged using 2×2 pixels. Each point automatically captures 5-color fluorescence channel images and 3 bright field channel images from different z-axis focal points. The acquisition of the phenotypic feature data of the test organisms with automatically captured images comprises positioning cells, nuclei, and cytoplasm in each image, and a quality of each image satisfies the following conditions: image intensity mean value of 10-240, image focus score>0.5, image edge front standard deviation<0.2, cell area of 50-500, cell debris hole number<5, cell density>50, and cell nucleus staining clarity>1.5; a gray level co-occurrence matrix for texture feature analysis was used to calculate a morphology, intensity, texture, brightness, average grayscale, a minimum distance between cells, adjacency values, and clustering degree, to obtain 5797 morphological feature items of each cell and an arithmetic mean of morphological feature values corresponding to the morphological feature items of each cell.
3. The collinear crossing feature terms of the phenotypic feature data of the algae cells and gill cells were excluded, and the feature items whose eigenvalues were not equal to 0 were retained; the standardizing feature values comprised a Z-Score method and a maximum-minimum method; the clustering arrangement was to classify and integrate the feature items according to corresponding subcellular structure of the feature items, and corresponding categories comprise algae cell DNA, algae cell endoplasmic reticulum, algae cell nucleosomes and cytoplasmic RNA, algae cell actin with Golgi apparatus and plasma membrane, algae cell chloroplasts, algae cell bright field, fish gill DNA, fish gill endoplasmic reticulum, fish gill nucleosomes and cytoplasmic RNA, fish gill actin with Golgi apparatus and plasma membrane, fish gill cell mitochondria, and fish gill cell bright field, to construct the toxicity matrix.
4. The machine learning model was built based on acute toxicity effect values and the phenotypic feature data of algae cells and acute toxicity effect values and the phenotypic feature data of the fish gill cells through a random forest model; feature dimensionality reduction was carried out on the constructed toxicity matrix using partial least squares discriminant analysis, to obtain 12 feature variables of whole water toxicity, which were substituted into the machine learning model to obtain the whole toxicity of the wastewater sample of the plant B, expressed as a 10% effect concentration (EC₁₀).

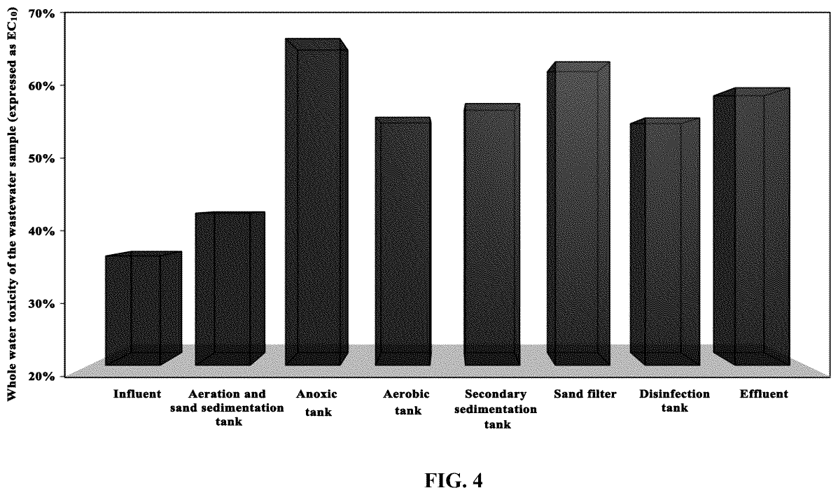

The subcellular structural images of algae cells and gill cells in the whole-wastewater sample of the plant B were obtained according to the method of the example. The cellular morphological features were extracted to obtain the cellular phenotypic feature data of algae cells and gill cells for construction of the toxicity matrix. The least partial squares discriminant analysis method was used to reduce the dimensionality of the toxicity matrix the whole-wastewater sample of the plant B, and 12 whole toxicity feature variables associated with water quality were obtained, which were substituted into the machine learning model to obtain the whole water toxicity of the influent, aeration and sand sedimentation tank, anoxic tank, aerobic tank, secondary sedimentation tank, sand filter, disinfection tank, and effluent from plant A, as shown in FIG. 4, which were 36.0%, 42.3%, 67.8%, 56.3%, 58.3%, 64.4%, 56.2%, and 60.6%, respectively.

### Example 3

Unlike Example 1, the application object of the example was effluent samples of three municipal wastewater treatment plants C, D, E in the Beijing-Tianjin-Hebei region, with a daily capacity of 1.2-2.8 million cubic meters per day, effluent containing 42.00-58.89 mg/L of COD, 6.26-10.09 mg/L of total nitrogen, and 0.09-0.35 mg/L of total phosphorus.
1. Wastewater samples from the three municipal wastewater treatment plants C, D, E were filtered through a 0.22 μm membrane filter for aqueous solutions, respectively.
2. Algae cells of Selenastrum capricornutum and gill cells of rainbow trout were used as test organisms, and were exposed for 24 h in the filtered influent sample. Hoechst 33342 dye, concanavalin A/Alexa Fluor 488 dye, SYTO 14 dye, phalloidin/Alexa Fluor 568 dye, and wheat gene agglutinin/Alexa Fluor 555 dye were mixed to prepare a first multiple fluorescent staining agent, and the algae cells were exposed in the first multiple fluorescent staining agent. Hoechst 33342 dye, concanavalin A/Alexa Fluor 488 dye, SYTO 14 dye, phalloidin/Alexa Fluor 568, wheat-germ agglutinin/Alexa Fluor 555 dye, and MitoTracker Deep Red dye were mixed to prepare a second multiple fluorescent staining agent, and the gill cells were exposed in the second multiple fluorescent staining agent. A high content cell imaging and analysis system was adopted for high throughput automatic acquisition of subcellular structure images of algae cells and fish gill cells of 8 parallel experiment groups inoculated in a well plate. The subcellular structure images of algae cells were obtained using a 63× immersion objective lens, and the subcellular structure images of fish gill cells were obtained using a 20× immersion objective lens; the excitation/emission wavelengths of the 5-color fluorescence channel used for automatic imaging of algae cells were DNA 376-398 nm/417-477 nm, ER 442-502 nm/503-538 nm, RNA 491-571 nm/573-613 nm, AGP 502-622 nm/622-662 nm, Cy5 588-668 nm/652-732 nm; the excitation/emission wavelengths of the 5-color fluorescence channel used for automatic imaging of fish gill cells were DNA 376-398 nm/417-477 nm, ER 442-502 nm/503-538 nm, RNA 491-571 nm/573-613 nm, AGP 502-622 nm/622-662 nm, Mito 588-668 nm/672-712 nm. Each well in the orifice plate was equipped with 9 (3×3) imaging field points, which were merged using 2× 2 pixels. Each point automatically captures 5-color fluorescence channel images and 3 bright field channel images from different z-axis focal points. The acquisition of the phenotypic feature data of the test organisms with automatically captured images comprises positioning cells, nuclei, and cytoplasm in each image, and a quality of each image satisfies the following conditions: image intensity mean value of 10-240, image focus score>0.5, image edge front standard deviation<0.2, cell area of 50-500, cell debris hole number<5, cell density>50, and cell nucleus staining clarity>1.5; a gray level co-occurrence matrix for texture feature analysis was used to calculate a morphology, intensity, texture, brightness, average grayscale, a minimum distance between cells, adjacency values, and clustering degree, to obtain 5797 morphological feature items of each cell and an arithmetic mean of morphological feature values corresponding to the morphological feature items of each cell.
3. The collinear crossing feature terms of the phenotypic feature data of the algae cells and gill cells were excluded, and the feature items whose eigenvalues were not equal to 0 were retained; the standardizing feature values comprised a Z-Score method and a maximum-minimum method; the clustering arrangement was to classify and integrate the feature items according to corresponding subcellular structure of the feature items, and corresponding categories comprise algae cell DNA, algae cell endoplasmic reticulum, algae cell nucleosomes and cytoplasmic RNA, algae cell actin with Golgi apparatus and plasma membrane, algae cell chloroplasts, algae cell bright field, fish gill DNA, fish gill endoplasmic reticulum, fish gill nucleosomes and cytoplasmic RNA, fish gill actin with Golgi apparatus and plasma membrane, fish gill cell mitochondria, and fish gill cell bright field, to construct the toxicity matrix.
4. The machine learning model was built based on acute toxicity effect values and the phenotypic feature data of algae cells and acute toxicity effect values and the phenotypic feature data of the fish gill cells through a random forest model; feature dimensionality reduction was carried out on the constructed toxicity matrix using partial least squares discriminant analysis, to obtain 12 feature variables of whole water toxicity, which were substituted into the machine learning model to obtain the whole toxicity of the effluent samples of three municipal wastewater treatment plants C, D, E, expressed as a 10% effect concentration (EC₁₀).

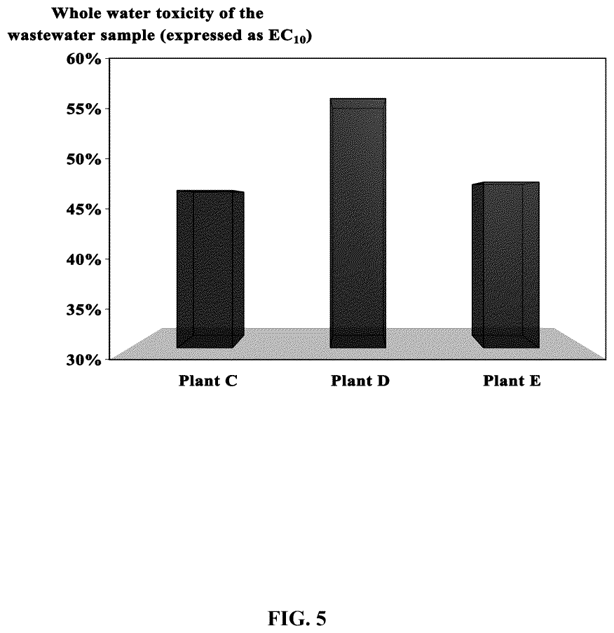

The subcellular structural images of algae cells and gill cells in the effluent samples of three municipal wastewater treatment plants C, D, E were obtained according to the method of the example. The cellular morphological features were extracted to obtain the cellular phenotypic feature data of algae cells and gill cells for construction of the toxicity matrix. The least partial squares discriminant analysis method was used to reduce the dimensionality of the toxicity matrix, and 12 whole toxicity feature variables associated with water quality were obtained, which were substituted into the machine learning model to obtain the whole water toxicity of the effluent samples of three municipal wastewater treatment plants C, D, E, as shown in FIG. 5, which were 47.0%, 56.9%, and 47.9%, respectively.

It will be obvious to those skilled in the art that changes and modifications may be made, and therefore, the aim in the appended claims is to cover all such changes and modifications.

What is claimed is:

1. A method for high-throughput determination of whole water toxicity, the method comprising:
1) exposing test organisms in a wastewater sample for pollution, obtaining phenotypic feature data of the test organisms;
2) constructing a toxicity matrix; and
3) building a machine learning model, and in combination with the toxicity matrix, determining a whole toxicity of the wastewater sample;
wherein:
following exposure in the wastewater sample for pollution, the test organisms are extracted through multiple fluorescent staining, high-content automated imaging, and cellular morphological characterization, to obtain the phenotypic feature data of the test organisms; and
the high-content automated imaging adopts a high content cell imaging and analysis system for high throughput automatic acquisition of subcellular structure images of algae cells and fish gill cells of 4-8 parallel experiments inoculated in a well plate.

2. The method of claim 1, wherein prior to exposing the test organisms in the wastewater sample, the wastewater sample is pretreated through a 0.22 μm membrane filter for aqueous solutions.

3. The method of claim 1, wherein the test organisms are algae cells and fish gill cells.

4. The method of claim 1, wherein multiple fluorescent stains for algae cell staining comprise Hoechst 33342 dye, concanavalin A/Alexa Fluor 488 dye, SYTO 14 dye, phalloidin/Alexa Fluor 568 and wheat-germ agglutinin/Alexa Fluor 555 dye; multiple fluorescent stains for gill cell staining comprise Hoechst 33342 dye, concanavalin A/Alexa Fluor 488 dye, SYTO 14 dye, phalloidin/Alexa Fluor 568 and wheat-germ agglutinin/Alexa Fluor 555 dye, and MitoTracker Deep Red dye.

5. The method of claim 1, wherein acquisition of the phenotypic feature data of the test organisms comprises positioning cells, nuclei, and cytoplasm in each image, and a quality of each image satisfies the following conditions: image intensity mean value of 10-240, image focus score>0.5, image edge front standard deviation<0.2, cell area of 50-500, cell debris hole number<5, cell density>50, and cell nucleus staining clarity>1.5; a gray level co-occurrence matrix for texture feature analysis is used to calculate a morphology, intensity, texture, brightness, average grayscale, a minimum distance between cells, adjacency values, and clustering degree, to obtain morphological feature items of each cell and an arithmetic mean of morphological feature values corresponding to the morphological feature items of each cell.

6. The method of claim 3, wherein the toxicity matrix comprises phenotypic feature data of algae cells and fish gill cells acquired through clustering arrangement after operations of filtering feature items and standardizing feature values; operation of filtering feature items is to exclude collinear crossing feature items, and retain feature items whose eigenvalues are not equal to 0; operation of standardizing feature values adopts a Z-Score method and a maximum-minimum method; the clustering arrangement is to classify and integrate the feature items according to corresponding subcellular structure of the feature items, and corresponding categories comprise algae cell DNA, algae cell endoplasmic reticulum, algae cell nucleosomes and cytoplasmic RNA, algae cell actin with Golgi apparatus and plasma membrane, algae cell chloroplasts, algae cell bright field, fish gill DNA, fish gill endoplasmic reticulum, fish gill nucleosomes and cytoplasmic RNA, fish gill actin with Golgi apparatus and plasma membrane, fish gill cell mitochondria, and fish gill cell bright field.

7. The method of claim 3, wherein the machine learning model is built based on acute toxicity effect values and the phenotypic feature data of algae cells and acute toxicity effect values and the phenotypic feature data of the fish gill cells, through a random forest model, XGBoost algorithmic model, Lasso regression algorithmic model, content-based recommendation algorithmic model, or support vector machine model.

8. The method of claim 1, wherein determining a whole toxicity of the wastewater sample comprises performing feature dimensionality reduction on the constructed toxicity matrix using partial least squares discriminant analysis, to obtain feature variables of whole water toxicity, and substituting the feature variables of whole water toxicity into the machine learning model to obtain the whole toxicity of the wastewater sample; the whole toxicity of the wastewater sample is acute toxicity effect values caused by the wastewater sample, and expressed as a 10% effect concentration (EC₁₀).
